SEQUENCE LISTING (4 sequences)

SEQ ID NO: 1 - DNA, 30 bp, Artificial Sequence
  Synthetic Oligonucleotide
acactctaagattttctgcatagcattaat

SEQ ID NO: 2 - DNA, 30 bp, Artificial Sequence
  Synthetic Oligonucleotide
attaatgctatgcagaaaatcttagagtgt

SEQ ID NO: 3 - DNA, 30 bp, Artificial Sequence
  Synthetic Oligonucleotide
attaatgctatgcggaaaatcttagagtgt

SEQ ID NO: 4 - DNA, 30 bp, Artificial Sequence
  Synthetic Oligonucleotide
attaatgctatgcaaaaaatcttagagtgt

(19) United States
(12) Patent Application Publication
(10) Pub. No.: US 2004/0005720 A1
(43) Pub. Date: Jan. 8, 2004

(54) Method and apparatus for temperature gradient microfluidics

(76) Inventors: Paul S. Cremer, College Station, TX (US); Hanbin Mao, College Station, TX (US); Tinglu Yang, College Station, TX (US)

Correspondence Address: Raymond Reese, Howrey Simon Arnold & White, 750 Bering Drive, Houston, TX, 77057-2198, US

(21) Appl. No.: 10/285,323

(22) Filed: Oct. 30, 2002

Related U.S. Application Data
(60) Provisional application No. 60/339,904, filed on Oct. 30, 2001.

(51) Int. Cl. G01N 33/53; G01N 33/543; C12M 1/00

(52) U.S. Cl. 436/518; 435/289.1; 435/7.1

(57) ABSTRACT

The present invention is an apparatus for providing a linear temperature gradient to an architecture suitable for massively parallel chemical or biochemical processing. The architecture is disposed on a substrate. The apparatus uses two temperature elements disposed essentially parallel to each other and in thermal contact with the substrate. When the temperature elements are held at different temperatures, a linear temperature gradient is formed in the substrate.

### CROSS REFERENCE TO RELATED APPLICATIONS

This application is a non-provisional application of U.S. Provisional Patent Application Serial No. 60/339,904 filed Oct. 30, 2001, the entire contents of which are incorporated herein by reference.

### FIELD OF THE INVENTION

The invention relates to methods and devices for controlling a temperature gradient across a apparatus for massively parallel chemical or biochemical analysis or synthesis. More specifically, a platform for high-throughput on-chip temperature gradient assays is described.

### BACKGROUND OF THE INVENTION

The advent of parallel data acquisition and combinatorial techniques has greatly expanded the experimental approaches employed in the biological and chemical sciences, leading to advances in areas ranging from genomics, proteomics, and small molecule screening to materials synthesis and catalyst optimization. Typical strategies rely on arraying many compounds on a two-dimensional grid such as a DNA chip or a multi-well plate. Variables such as buffer conditions, chemical composition and concentration can be easily controlled in a predetermined fashion at each address. Unfortunately, is not easy to probe temperature as a variable in a parallel fashion using combinatorial techniques. For example, it is impractical to supply a heating or cooling element to each well in a 96-well. A combinatorial approach to temperature dependent experiments would greatly benefit all of the areas mentioned above and would be particularly valuable for studying the crystallization of materials such as proteins and polymers, transition temperatures, and activation energies for chemical reaction.

### SUMMARY OF THE INVENTION

One aspect of the present invention is an apparatus for providing a linear temperature gradient to an architecture suitable for massively parallel chemical or biochemical processing. The architecture is typically disposed on a substrate, e.g., glass, poly(dimethylsiloxane) or silicon. The apparatus comprises first and second temperature elements disposed essentially parallel to each other and in thermal contact with the substrate. When the temperature elements are held at different temperatures, a linear temperature gradient is formed in the substrate.

A further aspect of the invention is a method of providing a linear temperature gradient to an architecture for massively parallel chemical or biochemical processing using an apparatus of the present invention. A still further aspect of the invention is a method of simultaneously determining the effect of temperature and at least one other parameter on the crystallization of an analyte using an apparatus according to the present invention.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A shows one embodiment of a temperature gradient microfluidic device.

FIG. 1B shows the geometry of the channels in the microfluidic device of FIG. 1A.

FIG. 2 is a schematic representation of a linear temperature gradient formed in the microfluidic device of FIG. 1.

FIG. 3 shows an alternative embodiment of a temperature gradient microfluidic device.

FIG. 4A shows a microfluidic device for two variable analysis.

FIG. 4B is an enlarged view of the mixing and loading regions of the device of FIG. 4A.

FIG. 5 shows a system of elastomeric, fluid-actuated valves for partitioning a microfluidic channel.

FIG. 6 shows temperature vs. position inside the microfluidic device of FIG. 1. Error bars for each point fit within the circles used to plot the data.

FIG. 7 shows temperature vs. position inside the microfluidic device of FIG. 3.

FIG. 8A shows a plot of the fluorescence of cadmium selenide nanocrystals in a pH 7.3, 10 mM phosphate buffer solution arrayed over a temperature gradient from 10 to 80° C. The particles were excited at 470 nm and emission was measured at 540 nm. The data were taken with an Eclipse 800 fluorescence microscope (Nikon). The variation in temperature across each of the 36 microchannels (cross section for each channel: 80 μm×7 μm) was less than 1.2° C. per microchannel.

FIG. 8B shows the same experiment as FIG. 8A run over a temperature range from 32.8 to 35.5° C.

FIG. 9A shows a plot of the percent recovery of fluorescence in a lipid bilayer after 1379 seconds for 14 parallel regions held at different temperatures using the fluorescence recovery after photobleaching technique.

FIG. 9B shows the recovery curves as a function of time for the data of FIG. 9A.

FIG. 10A is an Arrhenius plot of the dephosphorylation of 4-methylumbelliferyl phosphate to 7-hydroxy-4-methylcoumarin catalyzed by alkaline phosphatase immobilized in an array of 14 microchannels. The initial concentration of the substrate was 3.41 mM in a pH 9.8 sodium carbonate buffer with a total ionic strength of 150 mM.

FIG. 10B shows the reaction curves corresponding to FIG. 10A which were fitted by single exponentials of the form F=F₀+b(1−e^{-kt}) to obtain the values of k.

FIG. 11 shows a plot of fluorescence intensity of SYBR Green I dye vs. temperature in the presence of complementary DNA strands (triangles), DNA strands with a single T-G mismatch (filled circles), and DNA strands with a single C-A mismatch (open circles).

FIG. 12 shows a three-dimensional plot of fluorescence intensity of carboxyfluorescein dye molecules in aqueous solution as a function of their concentration (0.00715 to 0.266 μM) and temperature (28° C. to 74 ° C). The plot was mapped over 110 data points (excluded for clarity) gained from 11 temperature measurements across 10 microchannels. The grid intersections do not represent data points, but serve simply as a guide to the eye.

### DESCRIPTION OF ILLUSTRATIVE EMBODIMENTS

When heat flow is restricted to one direction along a two-dimensional planar surface, heat flow is governed by the Fourier heat diffusion equation (1):

$$\frac{ⅆ}{ⅆ x} ( k \frac{ⅆ T}{ⅆ x} ) = 0 \qquad ( 1 )$$

where T is temperature, x is the position along the direction of heat transfer, and k is the thermal conductivity of the medium in which the heat is flowing. If a hot reservoir and a cold sink are separated by a straight wall of thickness L within the plane, equation (1) can be doubly integrated to yield equation (2), which describes how the temperature inside the wall varies linearly between the two interfaces. In equation (2), T_{cold} is the temperature of the cold interface and Tₕₒₜ is the temperature of the hot interface. It is difficult to take advantage of equation (2) in macroscopic situations, but in the methods and devices of the present invention heat exchange in the third dimension is essentially negligible over the length-scales involved and linear temperature gradients can be achieved.

T(x)=T_{cold}+(Tₕₒₜ−T_{cold})×/L (2)

One aspect of the present invention is a apparatus for providing a linear temperature gradient to a substrate wherein the substrate comprises an architecture suitable for massively parallel chemical or biochemical processing. The apparatus comprises a first and second temperature element disposed essentially parallel to each other and in thermal contact with the substrate. The distance between the temperature elements is typically less than about 10 cm and more typically on the order of 10 μm to about 1 cm. When the distance between the temperature elements is to great, the linearity of the temperature gradient suffers.

As used herein, the term “architecture suitable for massively parallel chemical or biochemical processing” refers to any of the various architectures known in the art for manipulating very small volumes of fluid samples in a highly parallel fashion. One example is an array of wells, e.g., 96, 384, 1536, 6144 wells. These arrays are employed in combinatorial methods and are typically addressed using robotics. Another example is microfluidic systems which comprise channels or combinations of channels and reservoirs. Samples are typically manipulate in these devices using pressure or electrophoretic methods as describe in U.S. Pat. No. 5,904,824, the entire contents of which are incorporated herein by reference.

The architectures of the present invention typically comprise some means of containing fluid samples, e.g., wells, reservoirs, or channels. The volume of fluid contained in each well or channel is typically less than about 1 mL and more typically between about 10 μL and about 0.1 mL. Even smaller sample volumes (on the order of femtoliter-nanoliters) can be manipulated with embodiments of the present invention utilizing microfluidics. Small sample sizes have correspondingly low heat capacities. This is important in the present invention because it allows thermal equilibrium to be reached very quickly, e.g., as fast as 10⁷° C./s. As the volume of fluid increases, the heat capacity of the system also increases and thermal equilibrium is not reached as quickly. If the heat capacity becomes to great, heat flow in the third dimension will no longer be negligible, i.e., the assumptions contained in Equations (1) and (2) will no longer hold and linear temperature gradients will not be obtained.

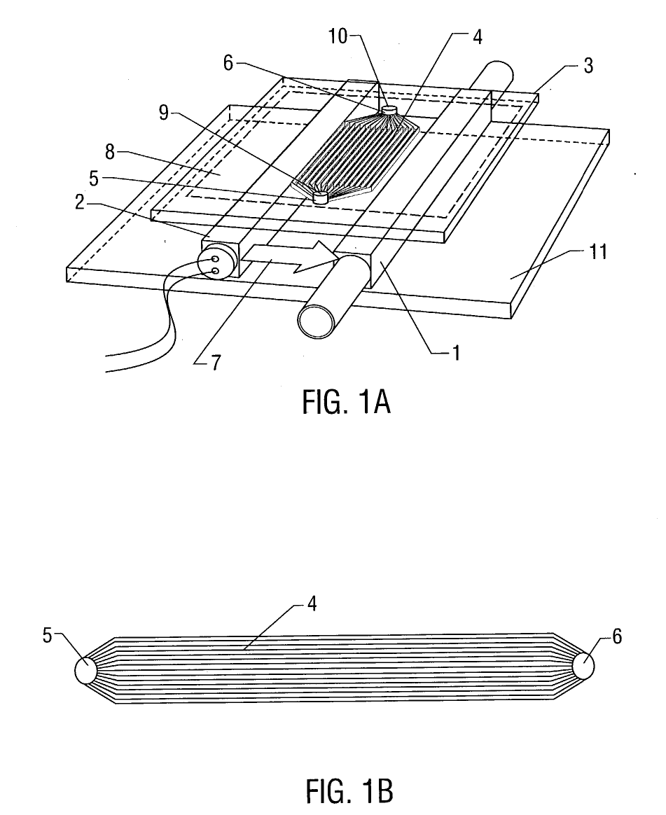

One embodiment of the present invention is shown in FIG. 1A and comprises first and second temperature elements 1, 2 and a substrate 3 in thermal contact with the temperature elements 1, 2. Temperature elements 1 and 2 can comprise a conduit for containing a fluid such as air, water, or a solution suitable for temperature control over a particular temperature range. The temperature element(s) 1 and/or 2 can be controlled, by controlling the temperature of the fluid, for example by using a circulating heating/cooling bath. A conduit type heating element can comprise any material that is thermally conductive and that is suitable for containing a fluid. Particularly suitable materials for the heating elements include brass, copper, and steel.

Alternatively, the first and/or second temperature elements 1, 2 can comprise an electrical heating element such as a heating cartridge, a resistively heated wire or filament, heating tape (e.g., NiCr tape), or a thermoelectric module (e.g., Peltier device). One of skill in the art will appreciate that some devices such as the thermoelectric module will operate more effectively in conjunction with a heat sink. In the embodiment depicted in FIG. 1A, temperature element 1 is a conduit for containing a temperature-controlled fluid and temperature element 2 is a heating cartrige.

The distance between the temperature elements can vary according to the size of the apparatus and the number of channels (discussed infra), but the distance should not be great enough to severely diminish the linearity of the temperature gradient, i.e., the distance should be such that equation (2) remains linear. The distance between the temperature elements is typically below about 10 cm and more typically below about 1 cm. According to one embodiment, the distance between the temperature elements is about 10 μm to about 15.0 mm. According to another embodiment, the distance is about 1.7 to about 2.3 mm.

The apparatus further comprises a substrate 3 in thermal contact with temperature elements 1 and 2. The substrate can be made of any material with sufficient thermal conductivity, that is chemically compatible with its intended purpose, and that is amenable to the fabrication of an architecture for massively parallel chemical or biochemical processing. Particularly suitable materials for the substrate include glass, poly(dimethylsiloxane), and silicon. Thermal contact between temperature elements 1, 2 and substrate 3 can be provided by direct physical contact or may be enhanced by an intervening, thermally conductive material. Examples of suitable thermally conductive materials include oil, grease, and water.

The substrate comprises a plurality of channels 4 disposed on the substrate 3. According to one embodiment, the channels can be etched into the substrate. The channels can be made using any available fabrication techniques, including lithographic techniques such as photolithography and soft lithography.

According to one embodiment, the channels 4 are disposed essentially parallel to each other. The length of the channels can vary depending on the application but is typically about 1 mm to about 40 mm, more typically about 8 mm to about 24 mm. Channels 4 typically have at least one cross sectional dimension that is about 10 to about 200 μm, more typically about 10 to about 50 μm. The space between channels can vary depending on the application but is typically about 10 to about 200 μm more typically about 50 to about 150 μm.

According to a particular embodiment, (shown more clearly in FIG. 1B) the channels emanate from a common origin 5 and terminate at a common terminus 6. This provides a convenient means of providing and removing analyte to all for the channels simultaneously.

In the embodiment shown in FIG. 1A, the channels 4 are disposed parallel to temperature elements 1 and 2. The temperature gradient 7 is therefore perpendicular to the channels. Each channel is at a unique position along the gradient and therefore at a slightly different temperature than the other channels. It should be noted that temperature gradient 7 is depicted schematically as extending between temperature elements 1 and 2 through space in FIG. 1A. This is for clarity only; in reality, heat flow occurs through substrate 3, between the areas of contact of 3 with elements 1 and 2.

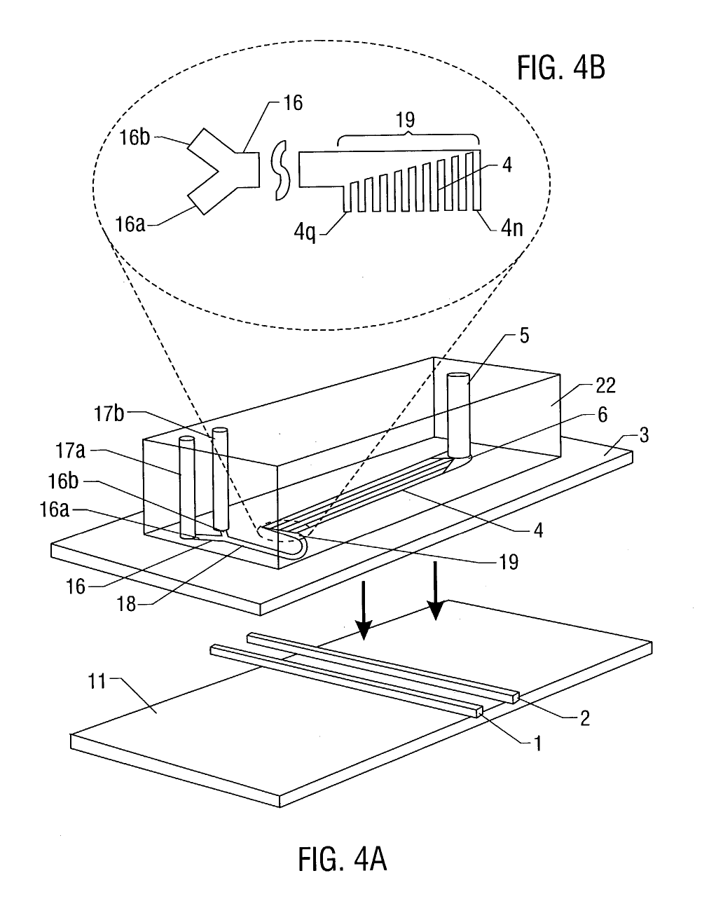

As shown in FIG. 4 (discussed in more detail below) the channels 4 can be disposed perpendicular to the temperature elements 1 and 2, i.e., parallel with the temperature gradient 7. According to this embodiment, each position along a given channel is at a unique temperature.

Referring back to FIG. 1A, the apparatus can further comprise a cover 8 disposed on the substrate 3. According to one embodiment the cover serves to seal off the plurality of channels 4. The cover can be made of any material that is chemically compatible with the intended use of the apparatus. Examples of suitable cover materials include glass and poly(dimethylsiloxane). According to one embodiment, the cover is optically transparent, thereby allowing optical or spectroscopic access to the channels. The cover can comprise inlet 9 and outlet 10 ports to provide analyte to and from the channels.

The apparatus depicted in FIG. 1A is disposed on a platform 11. The apparatus may be bound to the platform using any adhesion technique that is thermally stable within the range of temperatures to be applied by the temperature elements.

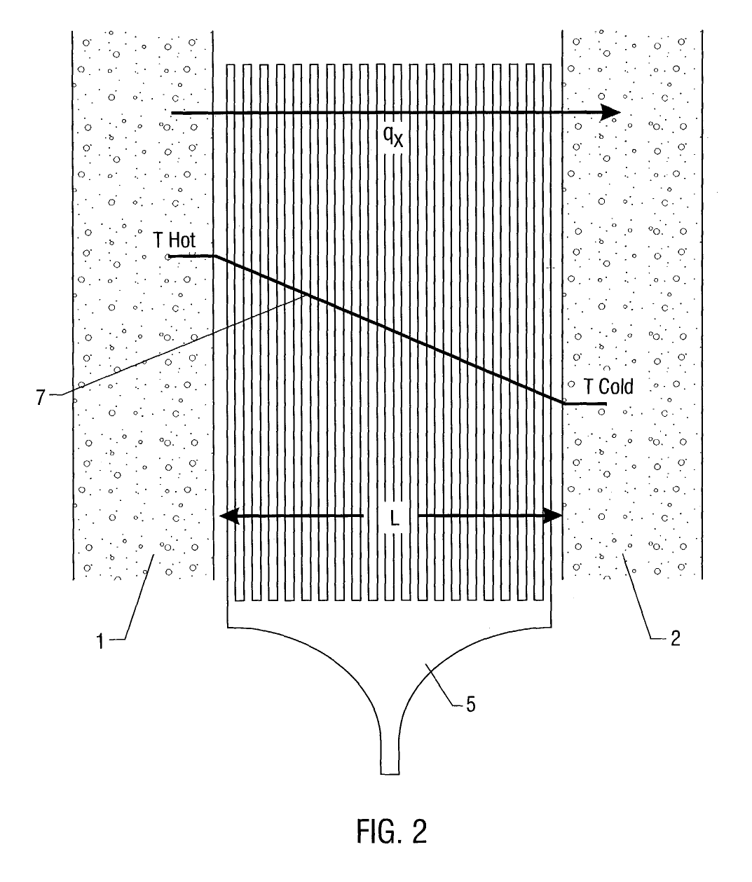

When differing temperatures are applied to temperature elements 1 and 2, for example a high temperature to 1 and a lower temperature to 2, a temperature gradient is formed between the elements according to equation (2). Such a temperature gradient 7 is depicted schematically in FIG. 2. The direction of heat flow is denoted by qₓ.

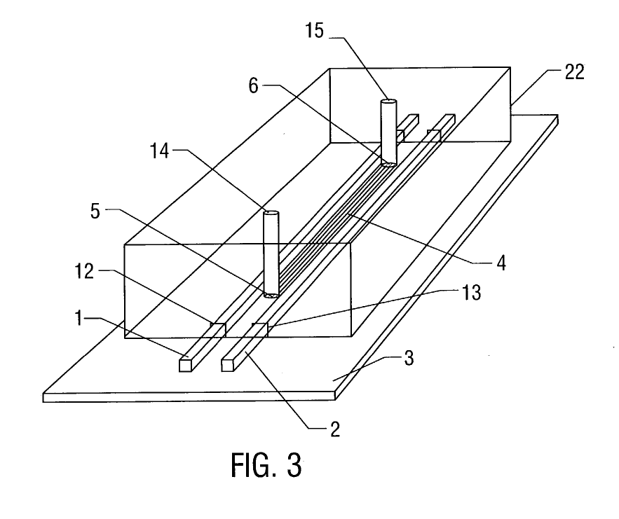

An alternative embodiment of the present invention is depicted in FIG. 3. It is an apparatus similar to the one described above but it includes a body 22 that comprises grooves 12, 13 for containing temperature elements 1 and 2. The plurality of channels 4 is etched into the body 22 and sealed by contact with the substrate 3. Access to the channels is provided by inlet and outlet ports 14 and 15, respectively, that pass through the body of the substrate 3. One of skill in the art will recognize that many alternative designs are possible, given the present disclosure, and are within the scope of the invention.

The temperature gradients provided by the above apparatuses are useful for a variety of applications. For example, phase transition temperatures of materials such as liquid crystals, membranes, and polymers can be investigated. When the channels are disposed parallel with the temperature elements, each channel will be at a different temperature. If the phase transition temperature is accompanied by a corresponding spectroscopic change, for example a change in fluorescence or absorbance, the channels can be interrogated through an optically transparent cover or substrate. According to one embodiment, a CCD camera is used to monitor the channels.

Chemical reactions can be monitored as a function of temperature by supplying the reactants to the channels, each of which is at a different temperature. The reactions can be monitored optically or if there is no convenient optical or spectroscopic observable for the particular process, the contents of the channels can be collected and analyzed using any applicable analytical technique, e.g. mass spectrometry, electrophoresis, or gas or liquid chromatography.

Temperature dependent monitoring of reactions is particularly useful for kinetics studies. The Arrhenius equation (equation (3)) can be used to determine the activation energy, Eₐ, for a chemical or biochemical reaction:

$$ln k = ln A - \frac{{E}_{a}}{RT} \qquad ( 3 )$$

In equation (3) k is the known rate constant for a reaction, A is a pre-exponential factor, T is temperature, and R is the gas constant (8.314 J/K-mol). Running the reaction at several different temperatures and plotting In k v. 1/T yields a line with a slope of −Eₐ/R and a y-intercept of 1 n A.

Monitoring the thermal transition between double stranded (ds) dsDNA and single stranded (ss) ssDNA is the principle diagnostic tool used in many DNA-based assays. For example, during PCR amplification, the melting curve of dsDNA is used to follow reaction progress and product purity. A single base pair mismatch reduces the amount of hydrogen bonding interactions in the ds species, therefore the transition temperature Tₘ of complementary dsDNA will be higher than the Tₘ of dsDNA with a mismatch. Although measuring DNA melting curves is essential for these techniques, current methods are hindered by the need to ramp the temperature sequentially. In PCR this is often done with a special thermal cycler.

Temperature gradients according to the present invention afford a convenient, one-shot method of obtaining a melting curve for dsDNA. An intercalation dye, for example SYBR Green I, is mixed with DNA samples and injected into a microchannel array. The experiment can be monitored using fluorescence microscopy. SYBR Green I is known to fluoresce when it is intercalated between stacked base pairs of dsDNA and to lose its fluorescence in aqueous solution. Therefore, a melting curve for dsDNA can be generated by monitoring for the loss of dye fluorescence as a function of temperature.

This method has several advantages compared to conventional DNA melting curve measurements. While standard techniques usually require at least hundreds of microliters and tens of minutes for a single curve, the present invention allows the same measurement with hundreds of nanoliters in just one shot (i.e. a few seconds). Because the fluorescence at all temperatures is detected simultaneously, the signal-to-noise ratio of the overall process is improved with respect to sequential analysis. This is because any variations in the light source intensity or detector yield as a function of time are avoided. Furthermore, the intercalation dye is subjected to far less photo and thermal damage due to the reduction in time of exposure to the excitation source and to temperature extremes. The geometry of this method can be adapted to acquire multiple DNA melting curves simultaneously by injecting different DNA strands into each channel and employing the strategy described below for multidimensional on-chip analysis.

FIG. 4A shows a still further embodiment of the apparatus, wherein channels 4 are perpendicular to temperature elements 1 and 2 and therefore parallel with the temperature gradient. The apparatus of FIG. 4 also comprises a means of mixing or diluting analytes as they are applied to the plurality of channels 4. Two streams of liquid merge at a Y-junction 16, shown in expanded view in FIG. 4B. Referring back to FIG. 4A, inlets 17ₐ and 17_{b} provide the streams to the Y-junction 16 where they merge and diffuse into each other as they flow downstream side by side through mixing region 18. Ideally, only diffusional mixing occurs because the Reynolds number inside mixing region 18 is low enough to prevent turbulence. The length of mixing region 18 can vary but is typically about 0.2 to about 4 cm. The greater the distance the liquids flow together, the more they are allowed to mix. The liquids then flow to loading region 19 where they are loaded into channels 4 as function of distance. Because only diffusional mixing occurs, the streams will vary in composition from 4ₐ to 4ₙ. For example, if component A is provided to 16ₐ and component B is provided to 16_{b}, then the composition in channel 4ₐ will be greater in component A because it does not have as much of a chance to mix with component B as analyte that proceeds further through loading region 19.

The embodiment depicted in FIG. 4 is a multidimensional assay because it allows the effect of temperature to be interrogated along one dimension of the apparatus and the effect of composition to be interrogated along a second dimension. Variables such as analyte concentration, pH, and buffer concentration can be varied from channel to channel and each probed simultaneously at different temperatures. For example, one can vary analyte concentration from channel to channel by providing a solution of analyte to 16ₐ and buffer or solvent to 16_{b}.

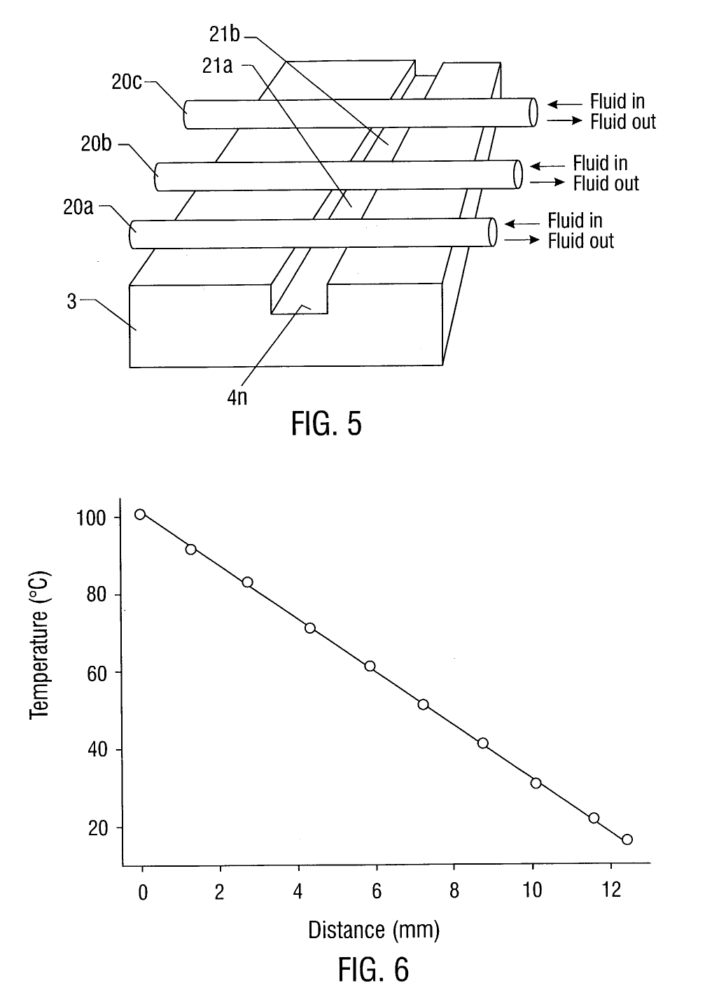

According to another embodiment of the present invention, the apparatus comprises channels that can be partitioned and into reservoirs that are hermetically sealed from each other. Several techniques exist in the art for partitioning microfluidic channels. For example, elastomeric, fluid-actuated valves are described in U.S. Pat. No. 6,408,878, the entire contents of which are incorporated herein by reference. FIG. 5 schematically depicts a representative channel 4ₙ disposed on substrate 3. Elastomeric tubes 20ₐ, 20_{b}, and 20_{c} are disposed across channel 4ₙ. In the “open” state, tubes 20ₐ, 20_{b}, and 20_{c} are essentially evacuated and analyte can flow freely through channel 4ₙ. The valves are actuated, i.e., “closed,” by charging tubes 20ₐ, 20_{b}, and 20_{c}, with sufficient fluid that they expand to block channel 4ₙ effectively isolating compartments 21ₐ and 21_{b} from each other. Because the analyte in channel 4ₙ is somewhat inelastic, it may be necessary to actuate the valves sequentially, i.e., 20ₐ followed by 20_{b} followed by 20_{c}, so that the analyte stream has the chance to equilibrate in response to increase in pressure due to the closing of the valves.

An alternative embodiment to those of FIGS. 4 and 5 is to simply replace substrate 3 comprising a plurality of channels with a substrate comprising an array of wells and using the platform-mounted temperature elements 1 and 2 to provide a temperature gradient across the array. Analyte can be added to the wells using any of the techniques known in area of combinatorial chemistry, for example robotics.

The multidimensional arrays having either actuated wells according to FIG. 5 or permanent wells are particularly valuable for studying protein crystallization. The crystallization of proteins are influenced by numerous factors including temperature, pH, protein concentration, and crystallization agent concentration. Also the presence and concentration of impurities or contaminants can effect crystallization. Because of the long time scales involved (days or months), the wells must be isolated from each other and be capable of being sealed. The multi-well embodiments of the present invention are therefore ideally suitable.

The following examples are included to demonstrate particular embodiments of the invention. It should be appreciated by those of skill in the art that the devices and techniques disclosed in the examples which follow represent those discovered by the inventor to function well in the practice of the invention, and thus can be considered to constitute some of the preferred modes for its practice. However, those of skill in the art should, in light of the present disclosure, appreciate that many changes can be made in the specific embodiments which are disclosed and still obtain like or similar results without departing from the spirit and scope of the invention.

### EXAMPLE 1

Glass microfluidic chip fabrication. Standard 50×75 mm soda lime glass slides (Corning) were cleaned by boiling in 7× detergent (ICN), rinsing with copious amounts of DI water and drying under a nitrogen stream. Photoresist (Shipley, S1813) was spun onto one side to a thickness of about 5 microns and soft baked for 1 hour at 90° C. in a convection oven. Photomasks were prepared by reducing a pattern printed with a 1200 dpi printer onto Kodak technical pan film. Samples were exposed using a Quintel 6000 mask aligner and developed in a 1:1 solution of Microposit developer concentrate (Microchem) and DI water. Slides were etched and bonded using a process adapted from Lin and coworkers. This involved gently waving photopatterned slides in a BOE (buffer oxide etchant) solution (1:6 ratio of 48% HF:200 g NH₄F in 300mL DI water) for 2.5 minutes, washing in a 1M HCl solution for 30 seconds and then placing the slides back into BOE for 2.5 minutes. This cycle of etching and washing could be repeated up to 6 times before the photoresist would degrade and peel away. The patterned lines were between 30 and 40 microns deep as determined by profilometry measurements. Chips were produced with 15 parallel microchannels that were 19 mm long, 120 microns wide, and spaced by 90 microns. At each terminus, the microchannels converged to a common 1 mm diameter outlet drilled into the glass using a diamond coated drill bit (Wale Apparatus). A 25.0×37.5 mm soda lime glass slide section was used as a cover for the microchannels. Covers and etched chips were cleaned by boiling in 7× detergent and then placed in a warm 6:1:1 DI H₂O:HCl:H₂O₂ solution for 5 minutes, a warm 5:1:1 DI H₂O:NH₄OH:H₂O₂ solution for 5 minutes, rinsed with copious DI water and finally dried with N₂. Each device was bonded by stacking the plates between weights in the following order: a 0.5″ thick solid brass substrate (which served as a base), a polished alumina flat, the etched chip (channels up), a soda lime glass cover, a second smaller polished alumina flat, and finally a 40 g brass weight. During thermal bonding, the weight would press on the alumina flat causing the two glass surfaces to fuse. After the initial firing, the weight and the flat would be moved to an unbonded section and the firing schedule rerun. This process was repeated until all vital areas were bonded. The firing sequence was as follows: From room temperature a 280 ° C./hr ramp was applied until 400° C. and held at that temperature for 4 hours. Next, a 280° C./hr ramp was applied up to 588° C. and held for 6 hours. Finally the kiln was shut off and allowed to cool to room temperature.

Temperature Gradient Apparatus. Initial temperature gradient characterization was done with a larger platform than described above and the bass tubes were separated by 12.6 mm. This device consisted of a PDMS (polydimethylsiloxane) mold bound to a planar glass surface. Channels were formed in PDMS by replica molding on a photoresist patterned surface. The PDMS surface was then rendered hydrophilic by oxygen plasma treatment and bonded to a glass slide. 10 holes were punched into the device using a syringe needle (300 μm i.d.) through the top of the PDMS mold down to the glass substrate. The device was placed on top of the brass tubes with the holes oriented along the temperature gradient such that the two holes on either end were directly above the metal tubes. A thermocouple was inserted into the holes to probe the temperature at each location, from which a plot of temperature vs. position was made for a range of 16° C. to 101° C. (FIG. 6). A small amount of vacuum grease was applied to the surface of each brass tube to ensure uniform contact to the microfluidic device. Identical experiments were performed with the standard gradient platform described above. These results were the same as the ones shown in FIG. 6, but only five holes were bored in parallel in a device because of the narrowness of the gradient.

### EXAMPLE 2

Fabrication of a linear temperature gradient device. An apparatus as depicted in FIG. 3 was formed by soft lithographic techniques. First, polydimethylsiloxane (PDMS) (Dow Coming Sylgard Silicone Elastomer-184, Krayden, Inc.) channels were formed by replica molding on a photoresist patterned surface onto which two 1/16th inch wide hollow square brass tubes had been laid in parallel and raised on 200 μm thick stints. The PDMS surface was then rendered hydrophilic by oxygen plasma treatment (PDC-32G plasma cleaner, Harrick Scientific, Ossining, N.Y.) and bonded to a glass coverslip. Glass cover slips, which served as floors of the microchannels, were cleaned in hot surfactant solution (ICN ×7 detergent, Costa Mesa, Calif.), rinsed at least 20 times in purified water from a NANO-pure Ultrapure Water System and then baked in a kiln at 400° C. for four hours before use. Sample materials in aqueous solution were flowed in through the inlet port using a Harvard PHD 2000 syringe pump (Harvard Apparatus, Holliston, Mass.), while hot and cold fluids were introduced through the brass tubing using standard waterbath circulators (Fisher Scientific, Pittsburgh, Pa.).

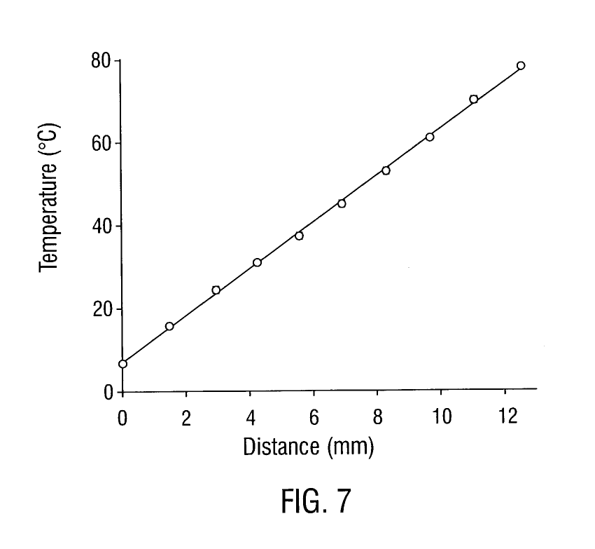

The temperature gradient in FIG. 7 was determined in the microfluidic device with 8 channels lying between the parallel heating and cooling tubes, which were separated by 12.6 mm. Using a syringe needle, 10 holes were drilled above the 8 channels and 2 metal tubes. The holes were formed in a line perpendicular to the brass tubes. A thermocouple (Omega Engineering, Inc., Stamford, Conn.) was used to probe the temperature at each location from which a plot of temperature vs. position was made.

A temperature distribution from 8° C. to 80° C. is shown in FIG. 7. It should be noted that the viscosity of water is about a factor of four greater at 8° C. than at 80° C. This means that the flow rate through the hottest channel was roughly four times faster than through the coldest channel. Since steady state temperatures are achieved extremely rapidly in microfluidic systems due to their very low heat capacity, this had no noticeable effect on the linear temperature distribution.

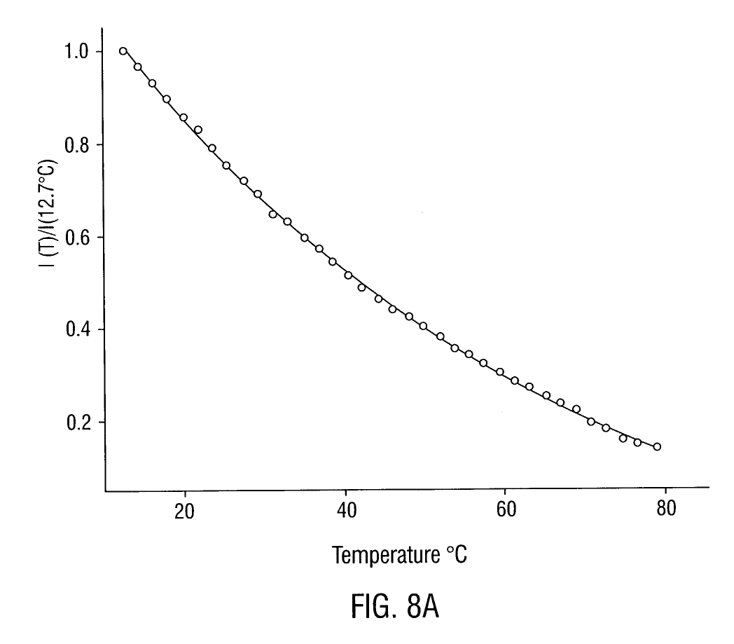

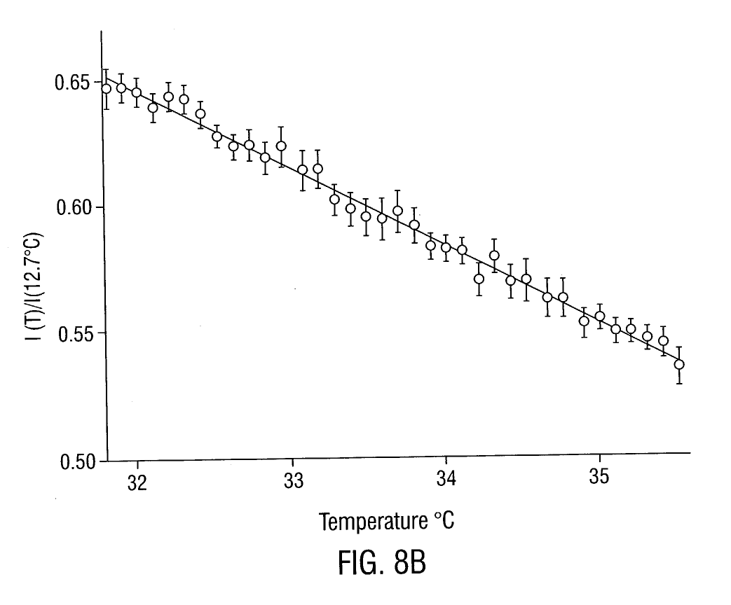

Fluorescence quantum yield of semiconductor nanocrystals. Soluble derivatives of semiconductor nanocrystals are receiving increasing attention because of their potential use as very bright, versatile fluorescent probes in biological systems. One notable physical characteristic of these particles is their highly temperature dependent fluorescence quantum yield. FIG. 8A shows the relative fluorescence yield of 8 nm diameter CdSe nanocrystals arrayed into 36 parallel channels with a temperature gradient from 10 to 80° C. The quantum yield varied by nearly an order of magnitude over this range and was somewhat nonlinear. On the other hand, an approximately linear dependence was observed when the experiment was performed over a sufficiently small range, as shown in FIG. 8B. In this case, the experiment was performed with a temperature gradient from 31.8° C. to 35.5° C., a separation of roughly 0.1° C. per data point. Obtaining data shallow temperature gradients requires that the heating and cooling elements be sufficiently stabilized against thermal drift.

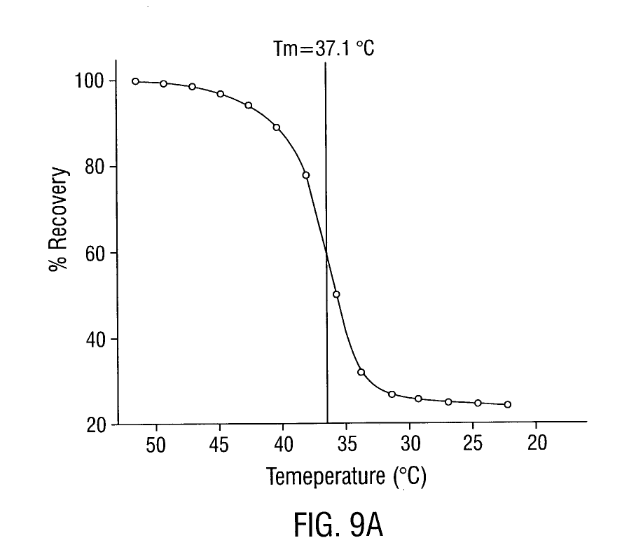

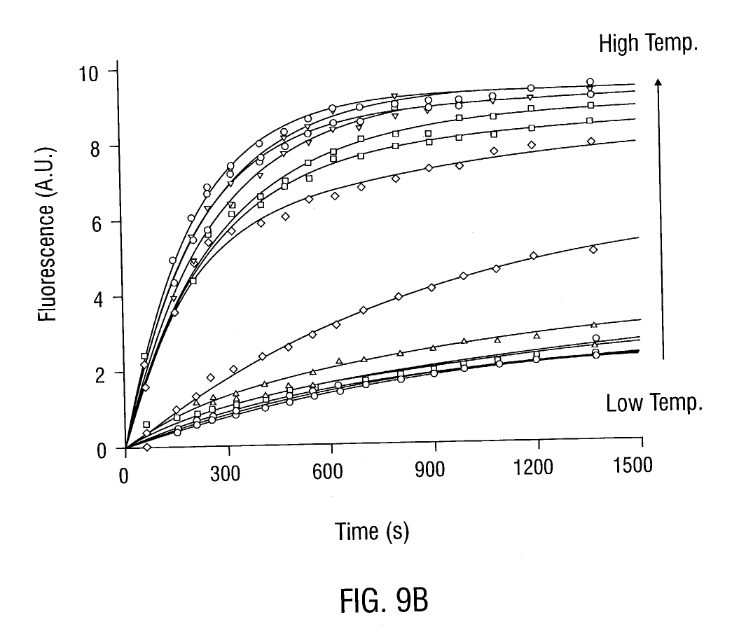

Phase transition measurement in a phospholipid membrane. The ability: to determine a phase transition temperature was demonstrated by measuring the main gel to liquid crystalline phase transition temperature for planar supported DPPC bilayers. The lipid membranes consisted of 99 mol % DPPC, a zwitterionic phospholipid with two 16-carbon chains, and 1 mol % of a fluorophore conjugated lipid, NBD-DPPE. Lipid bilayers were coated on the inside walls and floors of an array of 14 microchannels by the vesicle fusion method. The channels were situated in a temperature gradient from 22 to 51° C. and a line was then bleached simultaneously across the channels at time t=0. Because lipid bilayers are liquid crystals, the individual molecular components were constantly mixing in two-dimensions in the fluid phase, but mixing far more slowly in the gel phase. As a consequence, photo-oxidized NBD probes in the photobleached regions were replaced at different rates by fresh probes from the surrounding bilayer regions in the gel and liquid crystalline phases. This caused varying rates of fluorescence recovery in the initially darkened regions in each channel FIG. 9B. The percentage of recovery of each region after 1379 seconds is shown in FIG. 9A. While little more than 20% recovery was achieved by this point in time when the temperature was below 32° C., the value approached 100% above 45° C. The mid-point of the phase transition was near 37° C., in agreement with literature values.

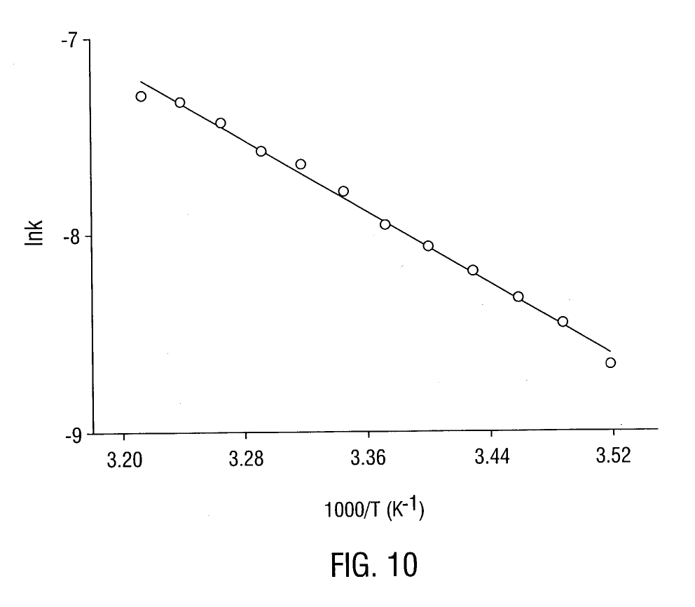

Activation Energy of a Phosphatase Enzyme. The activation energy Eₐ for the dephosphorylation of the non-fluorescent substrate, 4-methylumbelliferyl phosphate, to the highly fluorescent product, 7-hydroxy-4-methylcoumarin was determined. The dephosphorylation was carried out by the enzyme, alkaline phosphatase, which was immobilized on the walls and floors of the phospholipid bilayer coated microchannels by covalently linking it to the protein streptavidin and presenting 3 mol % biotinylated lipids in the membrane. Substrate was infused into the linear array of microchannels, mechanical valves at both ends were then shut, and the rate of product formation was directly monitored by fluorescence microscopy. FIG. 10 shows the results for a temperature gradient from 9 to 38° C. in 14 separate channels. The apparent activation energy of the reaction in this case was 38 kJ/mol, which is in good agreement with dephosphorylation rates of similar substrates.

### EXAMPLE 3

DNA melting curve measurements. Oligonucleotides (Integrated DNA Technologies, Coralville, Iowa) according to SEQ ID NOS. 1, 2, 3, and 4 were prepared at 113 nM concentration in Tris (Sigma) buffer solution at pH 8.0. After mixing 15 μL aliquots of complementary strands at equimolar ratio, the resulting mixture was heated to 94° C. for 5 minutes and allowed to cool to room temperature. 15 μL of SYBR Green I (Molecular Probes, Inc., Eugene, Oreg., 1:5000 dilution) in Tris buffer (pH 8.0) was added to the DNA solutions resulting in a final concentration of 56 nM for the dsDNA. The solution was incubated in the dark for 20 minutes before injection at room temperature into an all glass microchannel device. An all glass devices was employed for this experiment because PDMS-glass hybrid devices seemed to imbibe SYBR Green I dye and/or DNA into the polymer. After sample injection, the microfluidic device was brought into contact with the temperature gradient platform. The hot end of the stage was maintained at 77° C. and the cold end at 36° C. as verified by thermocouple measurements prior to every experiment. The fluorescence intensity was directly proportional to the signal intensity measured by our CCD camera, and it was therefore possible to relate the fluorescence signal to the degree of DNA melting. The fluorescence signal from the SYBR Green I was detected under a standard fluorescence microscope (E800, Nikon). The influence of temperature on the fluorescence of SYBR Green I was corrected by 2% per degree Celsius in accordance with standard literature procedures.

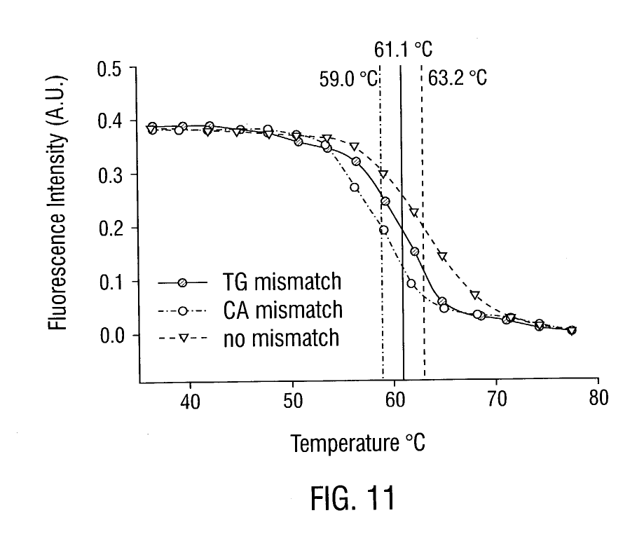

Single Nucleotide Polymorphism (SNP) Analysis. The intercalation dye, SYBR Green I, was mixed with DNA samples and injected into a microchannel array while fluorescence microscopy was performed. SYBR Green I is known to fluoresce when incorporated between stacked base pairs of dsDNA, but lose fluorescence in aqueous solution. Referring to the Sequence Listing below, SEQ ID NOS. 1 & 2 are complementary, while SEQ ID NOS. 1 & 3 contain a T-G mismatch and SEQ ID NOS. 1 & 4 have a C-A mismatch. Because a single base pair mismatch reduces the amount of hydrogen bonding interactions, the Tm of complementary dsDNA is higher than the Tₘ of dsDNA with a mismatch. This effect was observed in FIG. 11, where the melting curves of SEQ ID NOS. 1 & 2, 1 & 3, and 1 & 4 were 63.2° C., 61.1° C. and 59.0° C. respectively. These results matched expectations since T-G base pair interactions are known to be more stable than C-A base pair interactions. All curves were repeated several times with different devices and yielded essentially identical results each time.

### EXAMPLE 4

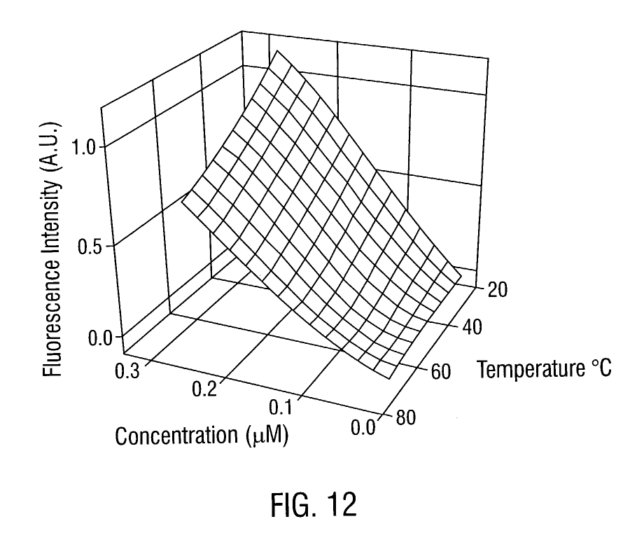

Multidimensional On-Chip Assay. A three-dimensional plot o fluorescence intensity of carboxyfluorescein dye in aqueous solution as a function of concentration and temperature was determined using an apparatus as depicted in FIG. 4. The apparatus was employed to create a dilution series that ranged over a factor of 37 in concentration in 10 parallel channels when the flow rate was set to 2 μl/min at each inlet. The fluorescein concentration injected at the inlet was 0.266 μM. A linear temperature gradient from 28° C. to 74° C. was established across the length of the channels after separation of the dye into the microchannels. After separating, the concentrations of fluorescein in each channel could be calculated relative to one another, because the fluorescence intensities of fluorescein were linearly related to concentration under the low concentration conditions employed. Even though the dye was constantly flowing through the microchannels, the temperature at any given point along a microchannel was considered to be at equilibrium. This assumption was deemed valid because small volumes of aqueous solutions in microchannels have been shown to equilibrate in similar local environments as fast as 10⁷ degrees ° C./sec. As can be seen from FIG. 12, the highest intensity was observed at the highest concentration and lowest temperature; however, the intensities varied in a complex manner. The two variable fluorescein assay demonstrates the potential of this technique to collect data in a massively parallel fashion. As with the single variable assay described above, this assay uses only low analyte volumes and provides excellent S/N, while effectively squaring the amount of data which can be collected.

All of the apparatuses and methods disclosed and claimed herein can be made and executed without undue experimentation in light of the present disclosure. While the apparatuses and methods of this invention have been described in terms of preferred embodiments, it will be apparent to those of skill in the art that variations may be applied to the apparatuses and methods described herein without departing from the concept, spirit and scope of the invention. All such similar substitutes and modifications apparent to those skilled in the art are deemed to be within the spirit, scope and concept of the invention as defined by the appended claims.

What is claimed is:

1. An apparatus for providing a temperature gradient to a substrate, the apparatus comprising:
a substrate having disposed thereon an architecture suitable for massively parallel chemical or biochemical processing;
first and second temperature elements disposed essentially parallel to each other wherein the first and second temperature elements are in thermal contact with the substrate and wherein the temperature gradient is essentially linear.

2. The apparatus of claim 1, wherein the architecture suitable for massively parallel chemical or biochemical processing is selected from the one or more of the group consisting of channels and an array of wells.

3. The apparatus of claim 1, wherein the architecture suitable for massively parallel chemical or biochemical processing comprises and array of wells, wherein the wells, are suitable for containing a fluid.

4. The apparatus of claim 3, wherein the array of wells comprises about 96 to about 384 wells.

5. The apparatus of claim 3, wherein the wells have at least one cross-sectional dimension of about 10 to about 50 μm.

6. The apparatus of claim 3, wherein each well is suitable for containing about 0.1 to about 100 μL of fluid.

7. The apparatus of claim 3, further comprising a protein disposed within at least one well.

8. The apparatus of claim 1, wherein the architecture suitable for massively parallel chemical or biochemical processing comprises at least two wells suitable for containing a fluid and at least one channel suitable for providing fluid communication between at least two wells.

9. The apparatus of claim 8, further comprising at least one valve, actuator, or pump suitable for manipulating a fluid.

10. The apparatus of claim 1, wherein at least one of the temperature elements is selected from the group consisting of a conduit for containing a temperature-controlled fluid, an electrical heating element, and a thermoelectric module.

11. The apparatus of claim 1, wherein the distance between the first and second temperature element is about 10 μm to about 1 cm.

12. An apparatus for providing a temperature gradient to a plurality of channels, the apparatus comprising first and second temperature elements disposed essentially parallel to each other, a substrate in thermal contact with the temperature elements, and a plurality of channels disposed on the substrate.

13. The apparatus of claim 12, wherein the plurality of channels is positioned at or above a point located between the temperature control elements.

14. The apparatus of claim 12, wherein at least one of the temperature elements is selected from the group consisting of a conduit for containing a temperature-controlled fluid, an electrical heating element, and a thermoelectric module.

15. The apparatus of claim 12, wherein at least one of the temperature elements comprises a conduit for containing a fluid.

16. The apparatus of claim 12, wherein at least one of the temperature elements comprises an electrical heating element selected from the group consisting of a resistively heated wire, a resistively heated tape, and a cartridge heater.

17. The apparatus of claim 12, wherein the substrate comprises a material selected from the group consisting of poly(dimethylsiloxane), glass, and silicon.

18. The apparatus of claim 12, wherein the substrate comprises poly(dimethylsiloxane).

19. The apparatus of claim 12, wherein the plurality of channels are disposed essentially parallel to each other.

20. The apparatus of claim 12, wherein the plurality of channels are disposed essentially parallel to the heating elements.

21. The apparatus of claim 12, wherein the plurality of channels are disposed essentially perpendicular to the heating elements.

22. The apparatus of claim 12, wherein the plurality of channels comprises from about 5 to about 50 channels.

23. The apparatus of claim 12, wherein the plurality of channels comprises channels having at least one cross sectional dimension of about 10 to about 50 μm.

24. The apparatus of claim 12, wherein the plurality of channels comprises channels having a length of about 10 μm to about 100 mm.

25. The apparatus of claim 12, further comprising an inlet for providing analyte to the plurality of channels and an outlet for removing analyte from the plurality of channels.

26. The apparatus of claim 12, further comprising two or more inlets for providing two or more streams to the plurality of channels, wherein the two or more inlets are disposed so that the two or more streams merge before they are provided to the plurality of channels.

27. The apparatus of claim 26, wherein the two or more inlets are disposed such that when the two or more streams merge before they are provided to the plurality of channels the streams mix together.

28. The apparatus of claim 27, wherein the merged streams are provided sequentially to each channel of the plurality of channels, and wherein the merged stream continue to mix as they are provided to the plurality of channels such that the merged streams are mixed to a greater extent as they are provided to each subsequent channel within the plurality channels.

29. The apparatus of claim 12, wherein the plurality of channels comprises channels that emanate from a common origin and terminate at a common terminus.

30. The apparatus of claim 12, wherein the plurality of channels comprises channels that are etched into the substrate.

31. The apparatus of claim 12, further comprising a cover disposed on the substrate.

32. The apparatus of claim 12, wherein the cover comprises a material selected from the group consisting of poly(dimethylsiloxane), glass, and silicon.

33. The apparatus of claim 12, further comprising a body disposed on the substrate.

34. The apparatus of claim 33, wherein the body comprises a material selected from poly(dimethylsiloxane), glass, and silicon.

35. The apparatus of claim 33, wherein the plurality of channels is etched into the body.

36. The apparatus of claim 12, further comprising at least one valve suitable for partitioning at least one channel of the plurality of channels into at least two hermetically sealed reservoirs.

38. The apparatus of claim 36, wherein the at least one valve is elastomeric valve.

39. A method for providing a temperature gradient to a substrate, the method comprising: thermally contacting the substrate with first and second temperature elements that are essentially parallel to each other; wherein the first temperature element is at a different temperature than the second temperature element; wherein the substrate comprises an architecture suitable for massively parallel chemical or biochemical processing; and wherein the temperature gradient is essentially linear.

40. The method of claim 39, wherein the architecture suitable for massively parallel chemical or biochemical processing is selected from the one or more of the group consisting of channels and an array of wells.

41. An method of claim 39, wherein at least one of the temperature elements is selected from the group consisting of a conduit for containing a temperature-controlled fluid, an electrical heating element, and a thermoelectric module.

42. An method of claim 39, wherein the distance between the first and second temperature element is about 10 μm to about 1 cm.

43. A method of simultaneously determining the effect of temperature and at least one other parameter on the crystallization of an analyte, the method comprising:
providing an apparatus, the apparatus comprising
a substrate, the substrate comprising an architecture suitable for massively parallel chemical or biochemical processing, wherein the at least one other parameter can be varied as a function of position on the substrate
first and second temperature elements disposed essentially parallel to each other wherein the first and second temperature elements are in thermal contact with the substrate,
varying the at least one other parameter as a function of position on the substrate, and
providing the first temperature element at a temperature that is different that the temperature of the second temperature element so that a linear temperature gradient is formed.

44. The method of claim 43, wherein the at least one other element is selected from the group consisting of analyte concentration, buffer concentration, pH, crystallization agent concentration, and the presence of impurities.

45. The method of claim 43, wherein the architecture suitable for massively parallel chemical or biochemical processing is selected from the one or more of the group consisting of channels and an array of wells.

46. An apparatus of claim 43, wherein at least one of the temperature elements is selected from the group consisting of a conduit for containing a temperature-controlled fluid, an electrical heating element, and a thermoelectric module.

47. An apparatus of claim 43, wherein the distance between the first and second temperature element is about 10 μm to about 1 cm.
